(19) United States
(12) Patent Application Publication
(10) Pub. No.: US 2023/0363699 A1
(43) Pub. Date: Nov. 16, 2023

(54) Method for classifying a polysomnography recording into defined sleep stages

(71) Applicant: Universitätsmedizin der Johannes Gutenberg-Universität Mainz, Mainz (DE)

(72) Inventors: Muthuraman Muthuraman, Freimersheim (DE); Haralampos Gouveris, Mainz (DE); Philipp Tjarko Boekstegers, Berlin (DE)

(21) Appl. No.: 18/029,149

(22) PCT Filed: Sep. 28, 2021
(86) PCT No.: PCT/EP2021/076608

§ 371 Date: Mar. 29, 2023

(30) Foreign Application Priority Data
Oct. 1, 2020 (DE) 10 2020 125 743.0

(51) Int. Cl. A61B 5/00; A61B 5/374

(52) U.S. Cl. CPC A61B 5/4812; A61B 5/374

(57) ABSTRACT

A method for classifying a polysomnography recording into defined sleep stages. The method comprises essentially the following steps: classifying the sleep of a human being into a grid with different sleep stages, collecting a plurality of information on bodily functions over a predetermined period of time in the form of data, wherein collecting the plurality of information comprises at least one measuring and recording of brain electrical activity data over a predetermined period of time during sleep of a person, subdividing the collected data into time-dependent data blocks, selecting a predetermined number of data blocks from the data blocks, wherein said data blocks include data on the electrical activity of the brain, automatically evaluating the brain electrical activity data in each selected data block using a cross-frequency coupling method, and automatically assigning the evaluated data blocks to a sleep stage.

### TECHNICAL AREA

The present invention relates to a method for classifying sleep stages based on a polysomnography recording. More particularly, the present invention relates to a method for classifying or categorizing a cardiorespiratory polysomnography recording into defined sleep stages.

### STATE OF THE ART

There are a large number of people who suffer from sleep disorders. Some of the sleep disorders are of a very different nature and can therefore have a variety of different causes.

It is well known that polysomnography recordings can provide clues to the causes of sleep disorders. Polysomnography records a variety of body function data from a patient during the sleep process. In particular, causes of sleep disorders can be identified from the progression of brain waves in specific areas of the brain, cardiac activity, and respiratory intensity and frequency during sleep. Therefore, during a polysomnography, brain waves at different locations of the brain are recorded by electroencephalography (EEG), e.g. according to the standards of the American Academy of Sleep Medicine (AASM) or according to Rechtschaffen and Kales, and cardiac activity is recorded by electrocardiography (ECG). In addition, respiratory parameters such as respiratory excursion of the thorax and abdomen, respiratory flow through the nose or mouth, and, if necessary, snoring sounds are recorded with the aid of a microphone, or electrical muscle activity on the chin as well as the lower legs is measured by means of electromyography (EMG).

Usually, polysomnography is performed in a specially equipped sleep laboratory.

Sleep is divided into five different stages based on current polysomnography standards, namely stage N1, stage N2 and stage N3 (as parts of non-REM sleep), REM stage, and awake stage, said awake stage corresponding to the epochs or period during sleep when the person is in the awake state. Physical activity respectively data on physical functions differ throughout these stages. This is noticeable, for example, in the fact that the brain waves, which are recorded by means of electroencephalography (EEG), are different in the individual stages. Among other things, both the frequency and the intensity of the brain waves differ.

In a healthy person, the sleep stages proceed in a more or less regular pattern. In patients with sleep disorders, this pattern may differ from that of a healthy person. In addition, depending on the absolute or percentage sleep stage classification during sleep, various bodily functions may deviate from those of a healthy person.

In order to find the cause of sleep disorders, it is therefore helpful to recognize the individual sleep stages of a patient and to assign bodily functions to certain sleep stages. The causes of a sleep disorder can be identified or better narrowed down on the basis of deviations found in the temporal sequence of the sleep stages and on the basis of deviations of individual bodily functions in the various sleep stages compared to a healthy person.

A polysomnography recording usually lasts seven to eight hours, as this is the usual duration of a person's sleep. Since some pathological events during sleep can last only a few seconds, the data are recorded at very short intervals, i.e. quasi continuously.

Due to the large amount of data, it goes without saying that the evaluation of such a polysomnography recording is very time-consuming. For the classification of the sleep stages of a polysomnography recording of a complete night alone, a specialist needs about one to two hours, with the sleep being divided into 30-second units, so-called epochs, wherein each epoch is assigned to a sleep stage. Furthermore, the quality of the classification depends on the experience of the specialist.

Attempts have been made to automatically classify a polysomnography recording. However, no satisfactory method has yet been found to automatically classify a polysomnography recording into different sleep stages with a high degree of accuracy.

### DESCRIPTION OF THE INVENTION

It is the object of the present invention to provide a method for classifying sleep stages of a polysomnography recording, which is as fully automatic as possible and reliably classifies a polysomnography recording into different sleep stages with high accuracy.

According to the invention, the object is solved by a method for classifying a polysomnography recording according to claim 1, which comprises the following steps:
First, the sleep of a human being is classified into grids with different sleep stages.
Then, a plurality of information is collected regarding bodily functions over a predetermined period of time in the form of data, wherein collecting the plurality of information comprises at least one measuring and storage of brain electrical activity data over a predetermined period of time during sleep of a person. The collected data is divided into time-dependent data blocks. This may be done manually, i.e. by a person, or preferably automatically by a computer or the like. Subsequently, manually, but preferably automatically, a predetermined number of data blocks is selected from the collected date blocks, with said date blocks containing information, in particular data on the electrical activity of the brain. In the next step, the brain electrical activity data in each selected data block is automatically evaluated by using a cross-frequency coupling method. Finally, the evaluated data blocks are assigned to a sleep stage.

With the help of the described method it is possible to classify the sleep stages in a polysomnography recording automatically, especially fully automatically. Until now, it was assumed that a classification of sleep stages with a very high accuracy is only possible with a semi-automated method, although there are also large differences in the accuracy of the classification in the semi-automated methods known so far. Completely surprisingly. It was found that the described method achieves an accuracy in classification that has previously only been achieved with very good semi-automated methods. Compared with many other previously known semi-automated methods, the results of the described fully automated method are even better.

Even if the procedure can be performed fully automatically, individual steps can still be performed manually. The decisive factor is that the assignment of selected data blocks to a sleep stage takes place automatically.

It has turned out to be particularly advantageous that the step of automatically evaluating the brain electrical activity data in each selected data block by means of a cross-frequency coupling method includes determining a characteristic value which allows an assignment to a sleep stage defined by the characteristic value. The determination of the characteristic value for a sleep stage can already be done before or independently of the recording of the bodily functions of a person (patient) during sleep.

The invention is based on the finding that brain waves measured by electroencephalography are particularly suitable for drawing conclusions about the sleep stage represented in a block of data. In particular, the C3/C4 data of the electroencephalogram are comparatively easy to determine. They belong to the data collected in the context of a polysomnography which are established in clinical practice worldwide and recorded according to all valid standards (e.g. standard according to Rechtsschaffen and Kales as well as according to AASM-American Academy of Sleep Medicine) and, due to their symmetrical arrangement on the head of a person, furthermore allow a comparison of the measurement results among each other. Therefore, according to a preferred embodiment of the described method, it is provided that measuring and documentation of brain electrical activity data is performed by means of an electroencephalography with measurement sensors, whereby preferably the measurement sensors of the electroencephalography are positioned on the skin of the skull surface. As indicated above, it is of particular advantage that the C3/C4 data of an electroencephalography are collected.

Furthermore, the invention is based on the realization that the data collected by means of an electroencephalogram result from a superposition of several oscillating signals. The electroencephalogram thus captures different frequency components that interact with each other. Classical analyses of power frequency, based for example on the (fast) Fourier transform (FFT) or various transforms of time (e.g., Hilbert transform), represent modulations of amplitudes within a defined frequency per time. However, they cannot identify the relationships of different frequencies or frequency components to each other. Using the cross-frequency coupling method, it is possible to synthesize coupling frequencies. Here, a cross-frequency coupling method that includes phase-amplitude coupling has proven particularly useful.

Thus, a cross-frequency coupling method that includes a phase-amplitude coupling is preferred.

It is known that different types of waves are superimposed in an electroencephalogram. Alpha, beta, gamma, delta and theta waves are distinguished in a known manner, which differ, among other things, in their frequency range. The amplitudes or the occurrence of the different waves depend on the activity of the individual person. In detail, alpha waves are assumed to be in the range of 8-13 Hz and occur during inactive wakefulness with eyes closed. Beta waves with a frequency of 14-30 Hz appear during mental activity. Gamma waves appear in the frequency range of 31-100 Hz at very high mental activity. Delta waves are in the frequency range of 1 to 3 Hz and indicate unconsciousness or deep dreamless sleep. Theta waves with a frequency of 4-7 Hz appear in stages of drowsiness or deep sleep.

Surprisingly, it was found that a classification performed by means of the described method has a particularly high accuracy when the cross-frequency coupling is applied to theta and gamma waves or to delta and alpha waves.

Using the cross-frequency coupling method, it is possible for a support vector machine to correctly classify comparable data with a high degree of certainty. According to a preferred further development of the method, the selected data blocks are transmitted as training data blocks to a support vector machine for creating a classification in the support vector machine, and at least a portion of the data blocks that were not selected as training data blocks are transmitted to the support vector machine and automatically classified into the known sleep stages.

To accurately evaluate the large number of existing data blocks in a short period of time, it is advantageous for the support vector machine to comprise an algorithm that uses a non-linear basis kernel function.

Regarding the evaluation of a polysomnography recording, it is advantageous that the recorded data are divided into a predefined time interval, wherein in particular the time interval is in the range of 15 seconds to 5 minutes and, in particular with regard to electroencephalographic signals, is preferably 30 seconds (so-called 30-second epoch).

In a preferred embodiment of the method, additionally data on the following bodily functions are recorded: cardiac activity, airflow of nasal and/or oral respiration, respiratory excursion of the thorax and abdomen, respiratory sounds, in particular snoring sounds, eye movement patterns, electrical muscle activity in the chin area as well as on the lower leg, wherein the data are preferably collected by means of the following measuring methods or measuring devices: electrocardiography, microphone, air flow meter, electromyography electrodes. This provides additional information on the state of health of a person.

By means of the described method it is possible to assign the data of this bodily function to certain sleep stages. Thus, comparatively easy statements can be made about anomalies and thus health problems.

In a first embodiment of the method, the data on the bodily functions may be collected in a sleep laboratory, wherein the data on the bodily functions are collected in the sleep laboratory preferably during the second night.

Alternatively, data on bodily functions can be collected in a home environment.

### BRIEF DESCRIPTION OF THE DRAWINGS

Preferred embodiments are explained in more detail with reference to the accompanying drawings, in which:

FIG. 1 shows a schematic representation of the flow of a semi-automated method for classifying a polysomnography recording into defined sleep stages based on electroencephalography (EEG) data in conjunction with a frequency coupling method;

FIG. 2 shows a schematic representation of the accuracy of classification of individual sleep stages using theta and gamma waves;

FIG. 3 shows a schematic representation of the accuracy of classification of individual sleep stages using delta and alpha waves.

### WAYS TO CARRY OUT THE INVENTION AND INDUSTRIAL APPLICABILITY

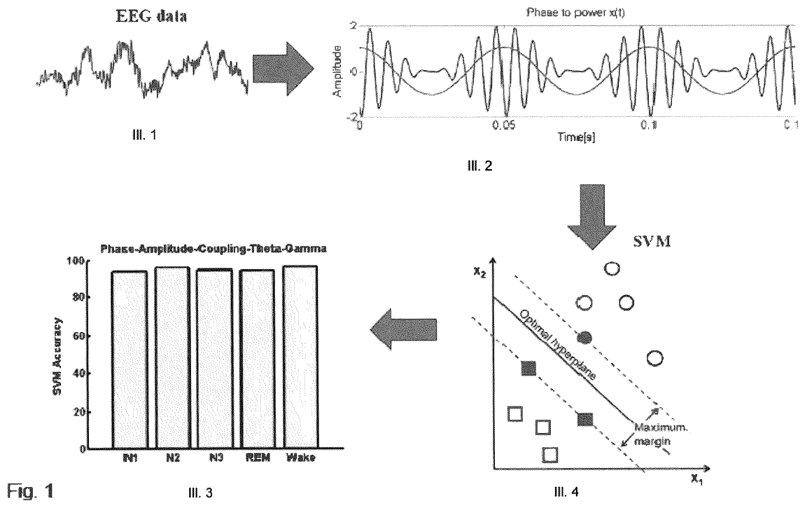

FIG. 1 shows a schematic representation of the flow of a semi-automated method for classifying a polysomnography recording into defined sleep stages based on electroencephalography (EEG) data in conjunction with a frequency coupling method.

In the first step of the method shown in FIG. 1, a person's sleep is divided into different sleep stages. Usually, sleep is divided into the five known stages, namely stage N1, stage N2, stage N3, REM stage and awake stage.

Each of these known stages can be identified on the basis of at least one data type. In this specific case, it is intended to automatically identify and classify the individual stages on the basis of the brain waves recorded by means of electroencephalography.

The next step is collecting a variety of information regarding bodily functions during a person's sleep in the form of a well-known polysomnography recording in a sleep laboratory. Typically, a polysomnography recording lasts seven to eight hours.

The collected data are divided into time-dependent data blocks with a duration of 30 seconds. This can be done manually, i.e. by a person, or automatically by a computer or the like.

From said data blocks, a trained person or a specialist selects a limited number of training data blocks and assigns each of these selected training data blocks to a sleep stage, wherein the person or the specialist selects the training data blocks in such a way that the data contained in the training block can each be uniquely assigned to a defined sleep stage. Ideally, the person or specialist selects the same number of training data blocks for each sleep stage. It has been shown that the selection of four training data blocks per sleep stage is sufficient. However, it goes without saying that more or fewer training data blocks can be selected within the scope of the described method.

The polysomnography recording and thus the data blocks contain, among other things, the brain waves recorded by means of electroencephalography. The brain waves were recorded at different locations in the brain. For the further procedure of classifying a polysomnography recording into sleep stages, the data recorded at positions C3 and C4 on the head of a patient by means of electroencephalography are used (see illustration 1 in FIG. 1). Positions C3, C4 are the positions commonly referred to as C3, C4 in electroencephalography.

The data of each training data block obtained at the C3/C4 positions of an electroencephalography are analysed using a data preparation procedure.

It is known that the frequency and amplitude of brain waves change during the different sleep stages. Each sleep stage is characterized by the presence respectively intensity or amplitude of different known frequency groups. Thus, the data displayed by the electroencephalogram at one position of the brain represent a superposition of different signals emitted by the brain in the form of brain waves. A simple frequency analysis of the collected data, for example in the form of a (fast) Fourier transform, due to the superimposed signals does not provide frequency sequences that can be clearly assigned to a sleep stage.

For this reason, the data obtained at the C3/C4 positions of the electroencephalography are processed using cross-frequency coupling (see illustration 2 in FIG. 1). Surprisingly, it has been found that a cross-frequency coupling method with a phase-amplitude coupling is particularly suitable for assigning sleep stages to the data of an electroencephalogram.

From the data collected in the course of electroencephalography, two frequency groups are identified at the C3/C4 positions, the course and intensity of which can be described precisely by means of phase-amplitude coupling. By phase-amplitude coupling, the dependence between the amplitude of a higher-frequency signal and the phase of a lower-frequency signal is represented. The characteristic course of the frequency groups processed by means of phase-amplitude coupling can be clearly assigned to a sleep stage.

The data of a data block obtained by means of cross-frequency coupling, in particular by means of phase-amplitude, are correlated with the sleep stage determined by a skilled person and thus form a training object.

The training objects obtained from the selected data blocks are transmitted to a support vector machine to create a classification in the support vector machine (see illustration 3 in FIG. 1).

An algorithm included in the support vector machine marks each data element as a point in n-dimensional space, where n represents the number of features. The algorithm has to calculate the best mean value between different separating straight lines in order to find the best common separating plane for all points, in this case a line with the maximum possible distance to all data points. The classification is performed by determining the so-called optimal hyperplane. As a next step, the algorithm looks for the hyperplane on which the data points with the smallest distance to said optimal hyperplane are located, the so-called support vectors. This distance is given the name Margin. The optimal separating hyperplane now maximizes the Margin to obtain clearly separated classification groups. The support vector machine thus divides the training data blocks into the specified sleep stages.

Then, the remaining data blocks that were not selected as training data blocks are transmitted to the support vector machine and an automatic classification of these data blocks into the known sleep stages based on the C3/C4 data of an electroencephalography is performed.

In a test phase, the described method was able to correctly assign the data blocks to sleep stages and thus achieve a hit rate of more than 93% (see illustration 4 in FIG. 1).

A particularly accurate classification of data blocks not selected as training data blocks is achieved by using a non-linear basis kernel function in the support vector machine algorithm.

Although it has previously been assumed that fully automated classification leads to comparatively poor results, in particular having significantly lower accuracy than semi-automated classifications, surprisingly high accuracy has been achieved in sleep stage classification using a fully automated method in which the cross-frequency coupling method was applied to EEG signals, in particular certain waves of the EEG signals, namely the C3 and C4 EEG signals.

In this fully automated method, as in the method shown in FIG. 1, the first step is to divide a person's sleep into different sleep stages. Usually, sleep is divided into the five known stages, namely the stage N1, the stage N2, the stage N3, REM stage and awake stage.

The next step is collecting a variety of information regarding bodily functions during a person's sleep in the form of a well-known polysomnography recording in a sleep laboratory, wherein information on the brain waves is among the information regarding bodily functions.

The brain waves can be recorded at different positions of the brain on the skin of the skull surface. However, for the further procedure of classifying a polysomnography recording into sleep stages, preferably the data recorded by electroencephalography at positions C3 and C4 on the skin of a person's (patient's) head are used. From the data at these two positions C3 and C4, in turn, the delta, alpha, gamma and theta waves are used for further processing or evaluation.

The collected data are divided into time-dependent data blocks with a duration of 30 seconds. This can be done manually, i.e. by a person, or preferably automatically by a computer or the like.

In order to classify the sleep stages, data blocks are selected as training data blocks and the theta and gamma waves or the delta and alpha waves of these training data blocks are processed using the cross-frequency coupling method. In particular, the corresponding phase-amplitude coupling of the cross-frequency coupling method is applied. The processed training data blocks are automatically assigned to the corresponding sleep stages.

The selected training data blocks processed by the cross-frequency coupling are transmitted to a support vector machine for creation of a classification in the support vector machine, as described in connection with FIG. 1.

After that, the remaining data blocks, which are not yet processed, are transmitted to the support vector machine and these data blocks are automatically classified into the known sleep stages based on the theta and gamma waves or the delta and alpha waves.

It is understood that with this method, all collected data blocks can be evaluated and processed using the cross-frequency coupling method, and these processed data blocks are then transmitted to the support vector machine.

Within the scope of the described method, it is useful to assign characteristic values to individual sleep stages before starting the method, wherein the characteristic values are obtained from the theta and gamma waves and the delta and alpha waves, respectively, which have been subjected to appropriate processing by the cross-frequency coupling method. From the theta and gamma waves or the delta and alpha waves of the data blocks, a value corresponding to the characteristic value of a sleep stage can then also be determined by means of the cross-frequency coupling method. With the help of the value obtained in this way, a corresponding classification, i.e. assignment of the data block to a sleep stage, can be carried out comparatively easily.

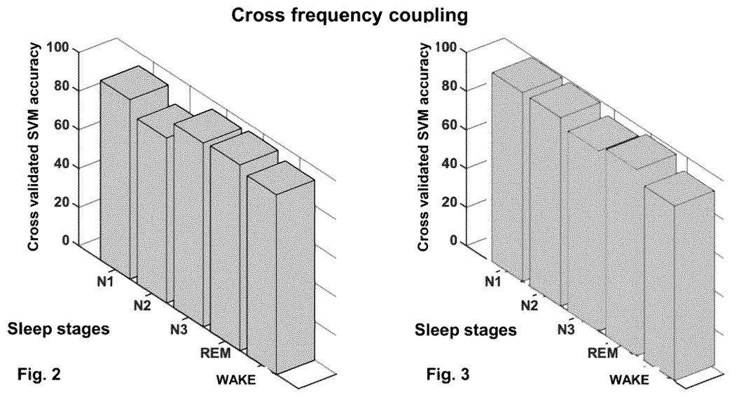

FIGS. 2 and 3 show a schematic representation of the accuracy of classifying individual sleep stages using theta and gamma waves (FIG. 2) and delta and alpha waves (FIG. 3), respectively, in a fully automated method with a cross-frequency coupling method that includes a phase-amplitude coupling.

With this fully automated procedure, an accuracy of classification for all sleep stages of more than 80% could be achieved, and for individual stages even more than 90%.

1. A method for classifying a polysomnography recording into defined sleep stages, comprising the following steps:
classifying the sleep of a human being into a grid with different sleep stages,
collecting a plurality of information regarding bodily functions over a predetermined period of time in the form of data, wherein collecting the plurality of information comprises at least one measuring and recording of brain electrical activity data over a predetermined period of time during sleep of the human being,
subdividing the collected data into time-dependent data blocks,
selecting a predetermined number of data blocks from the data blocks, wherein said data blocks include data on the electrical activity of the brain,
automatically evaluating the brain electrical activity data in each selected data block using a cross-frequency coupling method,
automatically assigning the evaluated data blocks to a sleep stage.

2. The method according to claim 1, characterized in that the step of automatically evaluating the brain electrical activity data in each selected data block by means of a cross-frequency coupling method includes determining a characteristic value that allows assignment to a sleep stage defined by the characteristic value.

3. The method according to claim 1, characterized in that measuring and documentation of the electrical activity of the brain is performed by means of electroencephalography with measurement sensors.

4. The method according to claim 3, characterized in that the C3/C4 data of an electroencephalography are collected.

5. The method according to claim 1, characterized in that the cross-frequency coupling method is applied to theta and gamma waves or to delta and alpha waves of an electroencephalography.

6. The method according to claim 1, characterized in that the cross-frequency coupling method comprises a phase-amplitude coupling.

7. The method according to claim 1, characterized in that the selected data blocks are transmitted as training data blocks to a support vector machine for creating a classification in the support vector machine, and that at least a portion of the data blocks not selected as training data blocks are transmitted to the support vector machine and automatically classified into the known sleep stages.

8. The method of claim 7, characterized in that the support vector machine comprises an algorithm that uses a non-linear basis kernel function.

9. The method according to claim 1, characterized in that the collected data are divided into a predefined time interval.

10. The method according to claim 1, characterized in that additionally data on the following bodily functions are recorded: cardiac activity, airflow of nasal and/or oral respiration, respiratory excursion of the thorax and abdomen, respiratory sounds, in particular snoring sounds, eye movement patterns, electrical muscle activity in the chin region as well as on the lower leg, wherein the data are preferably collected by means of the following measuring methods or measuring devices: electrocardiography, microphone, air flow meter, electromyography electrodes.

11. The method according to claim 10, characterized in that the additional data are evaluated as a function of the sleep stages.

12. The method according to claim 1, characterized in that the data on the bodily functions are collected in a sleep laboratory.

13. The method according to claim 1, characterized in that the data on the bodily functions are collected in a home environment.

14. A method for classifying a polysomnography recording into defined sleep stages, comprising the following steps:
classifying the sleep of a human being into a grid with different sleep stages,
providing a characteristic value for a sleep stage, wherein the characteristic value is determined from an EEG signal of an electroencephalography using a cross-frequency coupling method,
collecting a plurality of information on bodily functions over a predetermined period of time in the form of data, wherein collecting the plurality of information comprises at least one measuring and recording of brain electrical activity data on the skin of the skull surface by electroencephalography over a predetermined period of time during sleep of the human being,
subdividing the collected data into time-dependent data blocks;
selecting a predetermined number of data blocks from the collected data blocks, wherein said data blocks include brain electrical activity data in the form of EEG signals from the electroencephalography;
automatically evaluating the EEG signals using a cross-frequency coupling method and determining the characteristic value;
automatically assigning the evaluated data blocks to a sleep stage based on the characteristic value.

15. The method according to claim 3, wherein the measurement sensors of the electroencephalography are positioned on the skin of a skull surface.

16. The method according to claim 9, wherein the time interval is in the range of 15 seconds to 5 minutes.

17. The method according to claim 9, wherein the time interval is 30 seconds.

18. The method according to claim 12, wherein the data on the bodily functions are collected during a second night.
